(19) United States
(12) Patent Application Publication
(10) Pub. No.: US 2006/0269497 A1
(43) Pub. Date: Nov. 30, 2006

(54) Trichogenous agent

(75) Inventor: Yumika Okada, Shizuoka (JP)

Correspondence Address: LE-NHUNG MCLELAND, 9679-C MAIN STREET, FAIRFAX, VA, 22031-3766, US

(73) Assignee: YAMAHA HATSUDOKI KABUSHIKI KAISHA, Iwata-shi (JP)

(21) Appl. No.: 11/437,880

(22) Filed: May 22, 2006

(30) Foreign Application Priority Data
May 27, 2005 (JP) 2005-155452

(51) Int. Cl. A61K 31/12

(52) U.S. Cl. 424/70.1; 514/690

(57) ABSTRACT

According to the present invention, a trichogenous agent containing astaxanthin and/or an ester thereof is provided. The trichogenous agent of the present invention has a very low toxicity, and thus has a high degree of safety and can be used over a long period of time.

### BACKGROUND OF THE INVENTION

1. Field of the Invention

The present invention relates to a trichogenous agent having a high degree of safety.

2. Description of the Related Art

Where abnormalities relating to hair, such as alopecia, thin hair, hair loss, poliosis, or split hair, are concerned, since detailed mechanisms for hair development, hair growth, etc. have not yet been elucidated, various reasons have been proposed. For example, a heredity theory, a theory of imbalance of hormones and other such factors, a seborrhea theory, a theory of tension on scalp, a stress theory, and a sebaceous gland theory have been proposed. Accordingly, there are therapeutic or prophylactic methods (e.g., use of drugs) corresponding to the respective theories.

Hair restoring agents (hair restore agents) are roughly classified into trichogenous agents, hairgrowth promoters, agents for preventing hair loss, and anti-dandruff agents, in a broad sense. Among these, trichogenous agents refer to agents that, in the hair growth cycle in which anagen, catagen, and telogen phases are repeated, are highly effective in acting on, for example, trichogen cells in the telogen phase to induce hair to shift from the telogen phase to the anagen phase. Therefore, trichogenous agents are expressly as having a trichogenous effect, and are distinguished from common hair restoring agents. On the other hand, hairgrowth promoters act on trichogen cells in the anagen phase to delay the shift from the anagen phase to the catagen phase, thereby thickening hair, for example. Those products generally called hair restoring agents are hairgrowth promoters. Agents for preventing hair loss decrease hair in the telogen phase to reduce the falling out of hair; and anti-dandruff agents inhibit scalp inflammation.

As active ingredients for the trichogenous agents, only a limited number of compounds such as finasteride, which has a steroid backbone, and minoxidil, which is a pyrimidine-piperidine derivative, are known. On the other hand, as active ingredients for the hairgrowth promoters, a great variety of substances such as various galenicals, adenosine, flavan derivatives, and fatty acid derivatives are known. Between these trichogenous agents and hairgrowth promoters, no common features have been found in their chemical structures and biological activities.

Minoxidil, which is widely used in the world as a trichogenous agent, was developed as a vasodilator. Since the discovery of the trichogenous action of minoxidil as a side effect, minoxidil has been used as a trichogenous agent. However, use of minoxidil for a person who has an abnormality of the cardiovascular system is restricted. Moreover, finasteride which is a trichogenous agent also inhibits androgen, and thus its use for women is strictly prohibited.

Hair cosmetics such as hair restoring agents contain a variety of types of medicinal ingredients that are expected to provide a hair restoration effect. As the medicinal ingredients, for example, vitamins such as vitamin E, amino acids such as serine and methionine, vasodilators such as an acetylcholine derivative, anti-inflammatory agents such as a Lithospermum root extract, estrogen preparations such as estradiol, agents for enhancing the function of skin such as cepharanthin, agents for catalyzing melanin synthesis such as copper pantothenate, and keratolytics such as salicylic acid are included in hair cosmetics to prevent and treat alopecia. In some cases, a natural plant oil such as olive oil or castor oil or stearic acid is included to improve the properties of the resulting product. Moreover, hair cosmetics generally contain a higher alcohol or a derivative thereof.

Carotenoids (carotinoids), which are widely found in animals, plants, and microorganisms, are a class of about 600 different types of fat-soluble biopigments imparting a color ranging from yellow to orange to red. Astaxanthin, which is a carotenoid, is contained in, for example, crustaceans such as krils, shrimps, and crabs, muscles and eggs (salmon roe etc.) of salmon and trout, and the body surface of sea bream, carp, and goldfish. It is known that astaxanthin not only can become provitamin A and has a significant antioxidative effect but also has an anti-inflammatory effect (Japanese Laid-Open Patent Publication Nos. 7-300421 and 2004-331512, for example). The mechanism of action of astaxanthin is reported to be based on inhibition of the expression of inflammatory cytokines and chemokines (WO 01/072296), and inhibition of histamine release (U.S. Pat. No. 5,886,053).

A cosmetic containing astaxanthin has been disclosed, wherein astaxanthin is included mainly to stabilize a polyunsaturated fatty acid in the cosmetic by virtue of its antioxidative ability (Japanese Laid-Open Patent Publication No. 8-245335). Moreover, this application describes that astaxanthin shows promise of providing a pharmacological effect, and also discloses that astaxanthin aids in recovery from inflammation of biological cells, and helps to retard or prevent such inflammation of biological cells, as well as reduces the side effects of a drug, by preventing the production of lipid peroxide. Furthermore, use of astaxanthin for reducing hair loss, for restoring hair (i.e., strengthening hair, improving the properties of hair, and stimulating hair growth), and for preventing graying of hair has been disclosed (WO 03/105791), but there is no disclosure that astanxanthin has a trichogenous effect in which it induces the shift from the telogen phase to the anagen phase.

### SUMMARY OF THE INVENTION

The present invention provides a trichogenous agent containing astaxanthin and/or an ester thereof. Preferably, astaxanthin and/or an ester thereof is contained as an active ingredient.

The present invention also provides a method for regrowing hair comprising administering an effective dose of astaxanthin and/or an ester thereof to a subject.

In one embodiment, the administration is application to a site where hair is to be regrown. Preferably, the site where hair is to be regrown is scalp.

### BRIEF DESCRIPTION OF THE DRAWINGS

FIG. 1 is a graph showing the change over time in hair regrowth scoring at a site of application of an astaxanthin monoester in a test group, a positive control group, and a negative control group.

### DESCRIPTION OF THE PREFERRED EMBODIMENTS

Astaxanthin or an ester thereof used in the present invention is a carotenoid represented by the following formula: 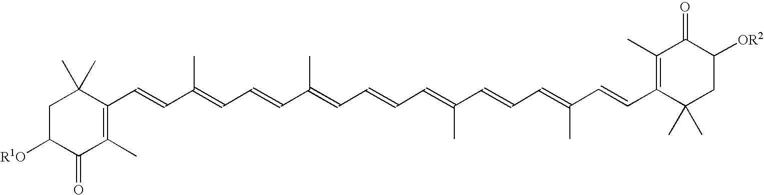
wherein R¹ and R² are both hydrogen in the case of astaxanthin, and R¹ and R² are each independently a hydrogen atom or a fatty acid residue provided that at least one of R¹ and R² is a fatty acid residue in the case of an ester of astaxanthin. Examples of the fatty acid residue in the ester of astaxanthin include, but are not limited to, saturated fatty acids such as palmitic acid and stearic acid or unsaturated fatty acids such as oleic acid, linoleic acid, α-linolenic acid, γ-linolenic acid, bishomo-γ-linolenic acid, arachidonic acid, eicosapentaenoic acid, and docosahexaenoic acid. The astaxanthin ester of the present invention can be any mono- or diester, homogeneous or non-homogeneous. Astaxanthin has a structure in which an additional oxo group and an additional hydroxy group are present at each end of a β-carotene molecule, so that unlike for β-carotene, the stability of the molecule is low. On the other hand, an ester form (e.g., as obtained in an extract from krill) in which the hydroxy groups at both ends are esterified with an unsaturated fatty acid is more stable.

Astaxanthin or an ester thereof used in the present invention may be chemically synthesized or derived from a naturally-occurring product. Examples of the naturally-occurring products in the latter case include red yeast; the shell of crustaceans such as Tigriopus (red water flea) and krills; and microalgae such as green algae, which contain astaxanthin and/or an ester thereof In the present invention, any extract containing astaxanthin and/or esters thereof produced by any method can be used. Generally, extracts from those naturally-occurring products can be used, and the extracts may be crude or purified if necessary. In the present invention, a crude extract or a crushed powder of naturally-occurring products, or a purified product or a chemically synthesized product, if necessary, that contains such astaxanthin and/or esters thereof can be used either alone or in combination. In view of the chemical stability, an ester form of astaxanthin is preferably used.

The trichogenous agent of the present invention preferably contains a higher fatty acid and/or a higher alcohol for their smoothing effect on hair and scalp. Examples of such a higher fatty acid and/or a higher alcohol include lauryl alcohol, myristyl alcohol, cetyl alcohol, stearyl alcohol, arachyl alcohol, behenyl alcohol, lauric acid, myristic acid, palmitic acid, stearic acid, and behenic acid. The higher fatty acid and/or the higher alcohol can be used alone or in combination as appropriate. There is no particular limitation on the amount of higher fatty acid and/or higher alcohol, but 0.5 to 10 (v/v) % based on the total amount of trichogenous agent is preferable and 1 to 8 (v/v) % is more preferable

The trichogenous agent of the present invention preferably contains a surfactant in that it imparts further softness to hair and skin. Examples of such a surfactant include cetyltrimethylammonium chloride, stearyltrimethylammonium chloride, behenyltrimethylammonium chloride, distearyldimethylammonium chloride, distearoylethyl hydroxyethylammonium methosulfate, dicocoylethyl hydroxyethylammonium methosulfate, N-[3-alkyl(12, 14)oxy-2-hydroxypropyl]-L-arginine hydrochloride, lauramide butyl guanidine acetate, N-cocoyl acyl-L-arginine ethyl.DL-pyrrolidone carboxylate, stearic acid dimethylaminoethylamide, and stearic acid diethylaminoethylamide. These surfactants can be used alone or in combination as appropriate. There is no particular limitation on the content thereof, but 0.1 to 5 (v/v) % based on the total amount of trichogenous agent is preferable and 0.3 to 3 (v/v) % is more preferable.

The trichogenous agent of the present invention may contain a blood circulation accelerator, a local irritant, and other ingredients in addition to the above-described ingredients. More specifically, blood circulation accelerators such as vitamin E and a derivative thereof, a swertia herb extract, a garlic extract, cepharanthin, carpronium chloride, and acetylcholine; local irritants such as capsicum tincture, cantharidis tincture, ginger tincture, and nonylic acid vanillyl amide; keratolytics such as salicylic acid, resorcine, and lactic acid; metabolic activators such as a placenta extract, glyceryl pentadecanoate, pantothenyl ethyl ether, biotin, hinokitiol, and allantoin; antiphlogistics such as glycyrrhizinic acid and glycyrrhetinic acid; bactericides such as isopropylmethylphenol, triclosan, zinc pyrithione, and hinokitiol; refreshing agents such as menthol and camphor; female hormones; and the like can be included. These ingredients may be used in combination as appropriate. Moreover, in the present invention, in addition to the aforementioned ingredients, other ingredients that generally can be included in hair cosmetics such as hair restoring agents, e.g., common cosmetic ingredients such as a lower alcohol, a polyhydric alcohol, a water-soluble polymer, an antioxidant, a pH regulator, an ultraviolet protective agent, a sequestering agent, a thickener, purified water, a perfume, an antiseptic, an antibacterial agent, an oil, a fatty acid ester, a humectant, a refreshing agent, and a pigment; or hormones, vitamins, amino acids, an astringent agent, and ingredients extracted from animals and plants such as a placenta extract, elastin, collagen, mucopolysaccharide, an aloe extract, the juice from the stem of Luffa cylindrica, royal jelly, birch, a ginseng extract, a chamomilla recutita extract, a glycyrrhiza extract, a sage leaf extract, a marshmallow root extract, and an Achillea millefolium extract, may be included, if necessary, as long as it does not impair the effects of the trichogenous agent of the present invention.

The trichogenous agent of the present invention is capable of promoting hair regrowth by inducing hair to shift from the telogen phase to the anagen phase. Promotion of hair regrowth can be confirmed, for example, by trimming away hairs in the telogen phase of the hair cycle and thereafter applying the trichogenous agent of the present invention to this region where the hairs have been trimmed away. More specifically, promotion of hair regrowth can be confirmed when the ratio of growing hairs, the hair growth rate, the proportion of hairs in the anagen phase per unit area, the hair diameter, and other hair properties are improved as compared to when the trichogenous agent is not applied.

There is no particular limitation on the amount of the trichogenous agent of the present invention to be applied. Generally, the trichogenous agent can be applied to an adult in a dosage of 0.1 mg to 500 mg expressed in terms of astaxanthin once to three times a day, preferably 1 mg to 50 mg once to twice a day.

### EXAMPLES

### Preparation Example 1

### Preparation of Astaxanthin Monoester

An astaxanthin monoester was prepared in the following manner. Haematococcus pluvialis K0084 strain was cultivated at 25° C under irradiation with light while bubbling a gas containing 3% CO₂ into the medium. Then, it was cultivated under nutrient stress condition (i.e. nitrogen source deprivation), and encysted. The encysted cells were disrupted by means commonly used by those skilled in the art, and a lipophilic fraction was extracted with ethanol. The extract contained lipids such as triglyceride in addition to astaxanthins. The extract was subjected to column chromatography using a synthetic resin adsorbent to give a purified product containing astaxanthin monoesters. This purified product was analyzed by HPLC, and it was confirmed that this purified product contained an astaxanthin monoester having a molecular weight of 858 as the main component, did not contain the free form of astaxanthin and the diester form of astaxanthin, and that it contained a small amount of diglyceride.

### Example 1

### Examination of Trichogenous Effect

The trichogenous effect in mice of the astaxanthin monoester obtained in Preparation Example 1 was examined. The astaxanthin monoester obtained in Preparation Example 1 was dissolved in 20% (v/v) ethanol-containing a-tocopherol to prepare a 10 mg/mL astaxanthin monoester test solution. Male 7-week-old C3H/HeN Slc (SPF) mice were divided into three groups, i.e., a test group, a positive control group, and a negative control group, of 10 each. It should be noted that 45 to 95-day-old C3H mice are in the telogen phase of the hair cycle. Hairs were shaved from a site measuring 5 cm x 3 cm on the back of each mouse, and the above-described test solution was applied to that site in a dosage of 0.05 mL once a day for 18 days to observe the conditions of hair regrowth. In the positive control group, 1% (w/v) minoxidil (in 50% (v/v) ethanol), and in the negative control group, 50% (v/v) ethanol were applied. The conditions of hair regrowth were evaluated, using the following scoring criteria, expressed in terms of percentage of the surface area of the test site where the observed change occurred.

0: skin was pink

1: skin changed to gray (in less than 50% of area)

2: skin changed to gray (in 50% or more of area) and hair regrowth was observed (in less than 50% of area)

3: skin changed to gray (in 50% or more of area) and hair regrowth was observed (in 50% or more but less than 80% of area)

4: hair regrowth was observed distinctly (in 80% or more but less than 100% of area)

5: hair regrowth was observed distinctly (in 100% of area)

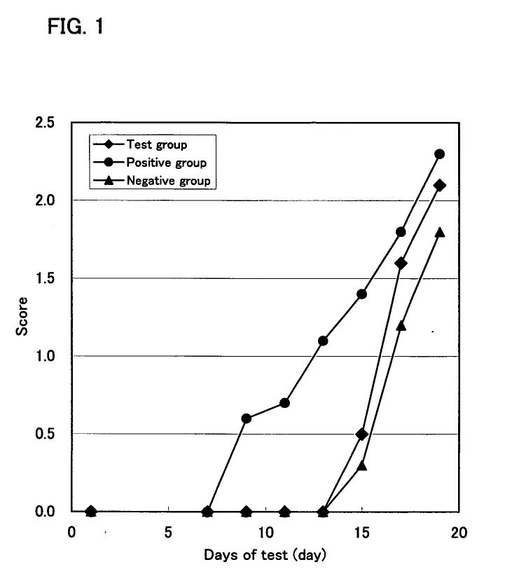

The results are shown in FIG. 1. In the test group, a markedly superior trichogenous effect was observed as compared to that in the negative control group. However, the trichogenous effect appeared a little later than in the positive control group. This suggests that the astaxanthin monoester exhibits its trichogenous effect slowly over a long period of time. Moreover, in the test group, though the initial hair regrowth speed was low, the rate of progress of hair regrowth after the hairs begun to grow was higher than that in the positive control group and, of course, higher than that in the negative control group. This is an effect specific to the test group.

### Reference Example 1

### Measurement of 50% Lethal Concentration for HUVEC

Human umbilical vein endothelial cells (HUVECs) (ATCC CRL-1730) were obtained from American Type Culture Collection and precultivated in an Endothelial Cell Growth Medium (CELL APPLICATIONS, USA) containing 10% bovine fetal serum supplemented with 1% Antibiotic-Antimycotic solution (GIBCO BRL, USA) under a 5% CO₂ atmosphere at 37° C.

A Matrigel matrix (BD Biosciences, USA) was melted and kept at 4° C. on ice, and then, 50 μL of the matrix were transferred to each well of a 96-well tissue culture plate. The plate was incubated at 37° C. for at least one hour to solidify the matrix solution.

On the other hand, the astaxanthin monoester obtained in Preparation Example 1 was dissolved in dimethylsulfoxide (DMSO), and then diluted with distilled water to prepare stock test solutions in which the astaxanthin monoester was contained in 40 (v/v) % DMSO at 25000, 2500, 250, 25, and 2.5 μM, respectively.

Next, 100 μL of a HUVEC suspension (about 2.5×10³ cells/well) were poured into the 96-well Matrigel plate under a 5% CO₂ atmosphere at 37° C. After 24 hours, 100 μL of a growth medium and 2 μL of each of the stock test solutions or the vehicle (40 (v/v) % DMSO) were added to two wells each, and incubated for an additional 72 hours. The final concentrations of the astaxanthin monoester were 250, 25, 2.5, 0.25, and 0.025 μM.

After the incubation, 20 μL of a 90% alamarBlue reagent were added to individual wells, and incubated for an additional 6 hours. Then, the fluorescence intensity of each well was measured at an excitation wavelength of 530 nm and an emission wavelength of 590 nm using a Spectrafluor Plus plate reader to count the number of living cells. This measurement is based on the ability of a living cell to change alamarBlue from the non-fluorescent, oxidized form (blue) to the fluorescent, reduced form (red). The 50% lethal concentration was calculated as the concentration at which the number of living cells was 50% of the number of cells at the start of the experiment.

The result indicates that the 50% lethal concentration (LC₅₀) of the astaxanthin monoester for the HUVECs was 250 μM (maximum concentration of the astaxanthin monoester dissolved in DMSO) or more, and thus it was found that the toxicity of the astaxanthin monoester is low.

According to the present invention, a novel trichogenous agent is provided. The trichogenous agent of the present invention is relatively slow-acting. However, having a very low toxicity, the trichogenous agent of the present invention has a high degree of safety and can be used over a long period of time.

The invention may be embodied in other forms without departing from the spirit or essential characteristics thereof. The embodiments disclosed in this application are to be considered in all respects as illustrative and not limiting. The scope of the invention is indicated by the appended claims rather than by the foregoing description, and all changes which come within the meaning and range of equivalency of the claims are intended to be embraced therein.

What is claimed is:

1. A trichogenous agent containing astaxanthin and/or an ester thereof.

2. A method for regrowing hair, comprising administering an effective dose of astaxanthin and/or an ester thereof to a subject.

3. The method of claim 2, wherein the administration is application to a site where hair is to be regrown.

4. The method of claim 3, wherein the site where hair is to be regrown is scalp.
